(19) United States
(12) Patent Application Publication
(10) Pub. No.: US 2021/0302918 A1
(43) Pub. Date: Sep. 30, 2021

(54) METHOD AND DEVICE FOR PROTECTING FROM ELECTRIC SHOCK BY DETECTING AND EVALUATING PHYSICAL-MEDICAL FACTORS

(71) Applicant: Bender GmbH & Co. KG, Gruenberg (DE)

(72) Inventor: Dieter Hackl, Fernwald (DE)

(21) Appl. No.: 17/213,318

(22) Filed: Mar. 26, 2021

(30) Foreign Application Priority Data
Mar. 26, 2020 (DE) 10 2020 108 390.4

(51) Int. Cl. G04G 21/02

(52) U.S. Cl. CPC G04G 21/02

(57) ABSTRACT

A method and a device for protecting a person from electric shock includes detecting a physical-medical factor by means of a sensor device (6) carried on the body (4) of the person; evaluating the physical medial factor, which has been detected by the sensor device (6) as sensor data (20), by means of an evaluation device (8) to discover whether a risk from a body current (I_{B}) is to be anticipated; and switching off a power supply (12), which is allocated to the person and causes the possible body current (I_{B}), by means of a switch-off device (10) if this risk exists.

This application claims the benefit of German Patent Application No. 10 2020 108 390.4, filed on Mar. 26, 2020, the disclosure of which is incorporated herein by reference.

### FIELD OF THE INVENTION

The invention relates to a method and a device for protecting a person from electric shock.

### BACKGROUND OF THE INVENTION

Statistical studies in Germany have shown that while on average the number of fatal accidents involving electricity has been declining in recent years as evidence of furtherly optimized protection and training measures when handling electricity at the workplace, the number of reported accidents involving electricity has been increasing. What is more, the studies reveal that over 90% of these reported accidents cite dangerous body currents as the cause.

The phenomenon of a decreasing number of fatal accidents involving electricity in combination with an increasing number of reported accidents involving electricity flowing through the body at physiologically critical levels as the main cause for the accidents involving electricity leads to the conclusion that protective measures and protective devices according to the state of the art show certain protection gaps.

A decisive protection gap persists in the difference between the electric sensitivity of humans with respect to electricity flowing through the body and the triggering sensitivity of residual-current devices according to the state of the art.

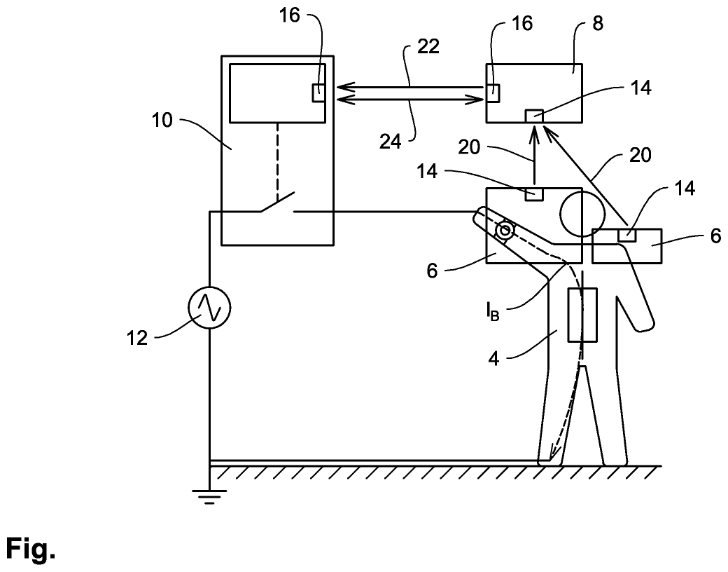

In a time/current diagram of physiological effects of body currents according to FIG. 20, the international standard IEC60479-1:2018 “Effects of current on human beings and livestock—Part 1: General aspects” cites a threshold curve b for AC currents on persons from 15 Hz to 100 Hz which flow between the left hand and the feet of a person; when these AC currents on persons are exceeded, impairing physiological effects are to be reckoned with. These effects occur above threshold line b until a threshold line c₁ in an AC-3 range and make themselves noticeable in the form of strong impulsive muscle contractions, difficulties when breathing or reversible heart defects, for example.

Threshold line b is surpassed when a body current of 5 mA or higher is attained and lasts longer than approximately 7 s. Above this threshold line b of 5 mA known as a let-go threshold, a person can no longer reliably extricate themselves from this body-current situation once muscle contractions have set in.

In contrast thereto, residual-current devices (RCD) have to safely break the circuit not before a nominal fault current of 30 mA has been reached according to product standard IEC61008-1 and must not break a circuit below a fault current of 15 mA to avoid false alarms.

This leaves a protection gap between the let-go threshold of 5 mA for current flowing through bodies and the breaking fault current of 15 mA or in the worst case even 30 mA for residual-current devices.

Besides the use of the residual-current devices mentioned above, using protective gear (work boots, gloves, plastic-sheathed tools) has been mandatory for specific applications to counteract this problem, and non-technical measures such as providing information and training and implementing safety protocols endeavor to reduce the number of accidents. However, these efforts for providing more safety and reducing the number of accidents are opposed by an increasingly more time and cost-efficient method of working, meaning the hitherto existing protective measures are dodged and safety protocols are ignored.

### SUMMARY

The object of the invention at hand is therefore to propose a method and a device for protection from electric shock which are both capable of predictive detection of potentially dangerous body currents and in particular of taking the protection gap for body currents between 5 mA and 15 mA or 30 mA into consideration.

This object is attained by a method for protecting a person from electric shock which comprises the following steps:
detecting a physical-medical factor by means of a sensor device carried on the body of a person;
evaluating the physical-medical factor, which was detected by the sensor device as sensor data, by means of an evaluation device to discover whether a risk from a body current is to be anticipated;
switching off the power supply allocated to the person and causing the body current by means of a switching-off device if this risk exists.

Advantageously, one or several of the following physical-medical factors are further detected: acceleration, heart rate, blood pressure, body temperature.

The fundamental idea of the invention at hand therefore rests on detecting physical-medical factors, such as acceleration of the human body or a body part, heart rate, blood pressure or body temperature, at the body of the person over a predetermined period of time, normally during a work assignment; evaluating these sensor data to discover whether a risk from a too high body current is to be anticipated; and if this risk exists, switching off the power supply which would cause the possible body current, i.e., the body current allocated to the acting person.

For the early detection of dangerous body currents and thus the prevention of risks, a coupling according to the invention takes place between detecting and evaluating body-related sensor data on the one hand (based on physical-medical factors) and a switching-off of the power supply takes place on the other hand.

The functional interaction starting with detecting a physical-medical factor, i.e., measuring bodily functions, with evaluating the sensor data and switching off the power supply allows detecting physiologically critical states for the individual person and to cause a selective automatic switching-off of the power supply as a consequence.

In another advantageous embodiment, the sensor data of the physical-medical factors detected by different sensor devices are merged.

Aiming to reliably detect a physiologically critical state ahead of time, the sensor data from different sensor devices are merged and evaluated together so as to support each other.

Preferably, the sensor data is evaluated for predictive detection of a possible body current residing above a normatively determined let-go threshold.

Evaluating the sensor data thus preferably targets early detection of a possible body current which could exceed the normatively determined let-go threshold of 5 mA.

Advantageously, the evaluation takes place using digital signal-processing algorithms.

For the early acquirement of an image of possible physiologically critical states from the detected and merged sensor data, digital signal-processing algorithms are implemented. The processing regulations can be algorithms for filtering, smoothing or predicting, for example, such as they are known from medial diagnoses for detecting artifacts. The evaluations can also be based on statistical signal descriptions to reduce errors in real measurement values and to provide estimates for non-measurable factors. Last, algorithms from the field of artificial intelligence can also be implemented.

Furthermore, the transfer of sensor data takes place via a wired or wireless connection using sensor-data transfer means.

If the sensor device and the evaluation device are not combined in an integrated structural unit, the sensor data can be transferred between the sensor device and the evaluation device via a wired cable connection path or via a wireless radio transmission path. For this purpose, a sensor-data transfer device comprises a send/receive part for data in the sensor device and the evaluation device, respectively.

Advantageously, the power supply is allocated and switched off via a wired or wireless connection using a switch-off-signal transfer device.

In order for the automatic selective switch-off of a power supply causing the possible body current to be effectuated, the power supply to be switched off can be allocated to the person at risk via a wired or wireless connection and switch-off signals can be transferred by means of the switch-off-signal transfer device via a wired or wireless connection.

In this instance, a send/receive unit for signals of the switch-off-signal transfer device disposed in the evaluation device and the switch-off device first sends identifying data to identify die power supply allocated to the person. Subsequently, a switch-off signal is transferred from the evaluation device to the switch-off device which in turn disconnects the power supply causing the possible body current.

In the case of a wireless transfer, standardized methods based on RFID, Bluetooth or WiFi technology are suitable.

The object of the invention at hand is further attained by a device for protecting a person from electric shock, the device comprising a sensor device carried on the body of the person for detecting a physical-medical factor; an evaluation device for evaluating the physical-medical factors detected by the sensor device as sensor data to discover whether a risk from a body current is to be anticipated; and comprising a switch-off device for switching off a power supply allocated to the person and causing the possible body current if this risk exists.

For implementing and executing the method according to the invention, the device according to the invention comprises a sensor device, an evaluation device and a switch-off device.

In this respect, the technical effects mentioned above in connection with the method and the advantages resulting therefrom also pertain to the device features.

In particular the device according to the invention comprises one or several sensor devices, such as an acceleration sensor, a heart rate sensor, a blood pressure monitor or a temperature sensor.

The sensor devices can be separate measuring instruments or, for example, be integrated in intelligent clothing (smartwear) or form a structural unit with other technical devices.

In particular the sensor devices and the evaluation device can be disposed in an integrated structural unit, for instance in the form of a smartphone or a wearable computer system known as wearables, e.g., smartwatches, fitness wristbands or digital glasses.

The evaluation device is preferably configured for merging the sensor data of the physical-medical factors detected by the different sensor devices for early detection of a possible body current residing above a normatively determined let-go threshold and for evaluating the sensor data using digital signal-processing algorithms.

Advantageously, the device according to the invention comprises a sensor-data transfer device for the wired or wireless transfer of the sensor data if the sensor device and the evaluation device are realized as structurally separate units.

The device according to the invention further comprises a switch-off-signal transfer device for allocating and switching off the power supply.

The switch-off-signal transfer device transfers identification data via a wired or wireless transfer method as well as a switch-off signal to the switch-off device.

### BRIEF DESCRIPTION OF THE FIGURE

Further advantageous embodiment features are derived from the following description and the drawing which describes a preferred embodiment of the invention using an example.

The FIGURE shows the device according to the invention in a functional illustration.

### DETAILED DESCRIPTION

The FIGURE shows a functional illustration of (protective) device 2 according to the invention in conjunction with a person through whose body 4 flows a body current I_{B} while working on an allocated power supply 12.

One or several sensor devices 6 record physical-medical factors, such as acceleration, heart rate, blood pressure or body temperature, and detect these as sensor data 20 in an electronically evaluable form.

Sensor devices 6 can be realized as autonomous sensors, e.g., electrodes, or in particular as an integral component of so-called wearables.

Sensor data 20 are merged in an evaluation device 8 and evaluated together to discover whether a risk from a possible body current I_{B} is to be anticipated.

For early detection of a possible physiologically critical state of human body 4 triggered by body current I_{B}, evaluation device 8 is configured for executing digital signal-processing algorithms and preferably comprises a microprocessor as a computing unit or computer. In particular the algorithms for evaluating sensor data 20 are programmed in such a manner that a body current is detected which could exceed the normatively determined let-go threshold of 5 mA.

Sensor device 6 and evaluation device 8 can form an integral structural unit and be carried on the person's body 4 as wearables, for example.

In an exemplary manner, sensor device 6 and evaluation device 8 are realized as separate devices in the FIGURE so that the transfer of sensor data 20 preferably takes place via a wireless connection using a sensor-data transfer device 14 or transmitter which comprises a send/receive part for data disposed in sensor device 6 and evaluation device 8, respectively.

If the evaluation of sensor data 20 in evaluation device 8 points to any indication of a physiologically critical state, evaluation device 8 forwards a switch-off signal 22 to a switch-off device 10 or switch allocated to the person in order to switch off allocated power supply 12.

In this example, switch-off signal 22 is transferred wireless by means of a switch-off-signal transfer device 16 which comprises a send/receive unit or transmitter for signals disposed in evaluation device 8 and switch-off device 10, respectively.

In order to allocate the acting person to the power supply system 12 to be switched off, additional identifying data 24 are transferred between evaluation device 8 and switch-off device 10 via switch-off-signal transfer device 16.

For this purpose, the wireless transfer method used in switch-off-signal transfer device 16 can be based on known methods, such as RFID, Bluetooth or WiFi.

1. A method for protecting a person from electric shock, the method comprising the steps:
detecting a physical-medical factor by a sensor (6) carried on the body (4) of the person,
evaluating the physical medial factor, which has been detected by the sensor (6) as sensor data (20), by a computer (8) to discover whether a risk from a body current (I_{B}) is to be anticipated, switching off a power supply (12) allocated to the person and causing the possible body current (I_{B}) by the switch (10) if this risk exists.

2. The method according to claim 1, wherein one or several of the following physical-medical factors are detected: acceleration, heart rate, blood pressure, body temperature.

3. The method according to claim 1, further including merging the sensor data (20) of the physical-medical factors detected by different sensors (6).

4. The method according to claim 1,wherein the sensor data (20) is evaluated for predictive detection of a possible body current (I_{B}) residing above a normatively determined let-go threshold.

5. The method according to claim 1, wherein the evaluation takes place using digital signal-processing algorithms.

6. The method according to claim 1,wherein the transfer of sensor data takes place via a wired or wireless connection using a sensor-data transmitter (14).

7. The method according to claim 1, wherein the allocation and switching-off of the power supply (12) takes place via a wired or wireless connection using a switch-off transmitter (16).

8. A device (2) for protecting a person from electric shock, the device (2) comprising: a sensor carried on the body (4) of the person for detecting a physical-medical factor, a computer (8) for evaluating the physical-medical factor detected by the sensor (6) as sensor data (20) to discover whether a risk from a body current (I_{B}) is to be anticipated,
a switch (10) for switching off a power supply (12) allocated to the person and causing the possible body current (I_{B}) if this risk exists.

9. The device (2) according to claim 8, including one or several of the following sensors (6): acceleration sensor, heart rate sensor, blood pressure monitor, body temperature sensor.

10. The device (2) according to claim 8,wherein the computer (8) is configured for merging the sensor data (20) of the physical-medical factors detected by different sensors (6).

11. The device (2) according to the claim 8, wherein the computer (8) is configured for predictive detection of a possible body current residing above a normatively determined let-go threshold.

12. The device (2) according to claim 8, wherein the computer (8) is configured for evaluating the sensor data (20) using digital signal-processing algorithms.

13. The device (2) according to claim 8, wherein a sensor-data transmitter is provided (14) for a wired or wireless transfer of the sensor data (20).

14. The device (2) according to claim 8, wherein a switch-off-signal transmitter (16) for allocating and switching off the power supply (12) by a wired or wireless transfer method.
